(19) United States
(12) Patent Application Publication
(10) Pub. No.: US 2011/0021483 A1
(43) Pub. Date: Jan. 27, 2011

(54) Platinum (IV) Complexes

(75) Inventor: Jan Gysbert Hermanus Du Preez, Port Elizabeth (ZA)

Correspondence Address: MORRISON & FOERSTER LLP, 755 PAGE MILL RD, PALO ALTO, CA, 94304-1018, US

(73) Assignee: PLATCO TECHNOLOGIES (PROPRIETARY) LIMITED, Pert Elizabeth (ZA)

(21) Appl. No.: 12/665,708

(22) PCT Filed: Jun. 18, 2008
(86) PCT No.: PCT/IB2008/052395

§ 371 Date: Jul. 15, 2010

Related U.S. Application Data
(60) Provisional application No. 60/929,228, filed on Jun. 18, 2007.

(51) Int. Cl. A61K 31/555; C07F 15/00; A61K 31/282; C12N 5/07; A61P 35/00

(52) U.S. Cl. 514/184; 556/137; 548/104; 514/492; 435/375

(57) ABSTRACT

Provided are nitroplatinum(IV) complexes containing a bidentate dicarboxylate ligand (e.g., oxalate) which maybe useful treating various forms of proliferative diseases, such as cancer. In some instances the platinum(IV) complexes are relatively stable and may be suitable for oral administration. Also provided are methods of treatment, as well as kits and unit dosages.

### CROSS REFERENCE TO RELATED APPLICATIONS

This application claims priority benefit of U.S. Provisional Application No. 60/929,228, entitled “Platinum(IV) Complexes” filed Jun. 18, 2007, the content of which is hereby incorporated by reference in its entirety as if it was put forth in full below.

### FIELD OF THE INVENTION

The present invention relates to novel platinum(IV) complexes which demonstrate potential anticancer activity.

### BACKGROUND OF THE INVENTION

Platinum complexes, such as cisplatin and oxaliplatin, have been widely used for the treatment of a variety of cancers. Since their development, great effort has been directed to the discovery of next-generation platinum complexes that have desirable properties, such as effective anticancer activity and/or reduced toxicity and undesired side effects.

Mononitro compounds of platinum(IV) (e.g., PtCl₃(NO₂)(NH₃)₂) have been described in, for example, (a) Chemyaev, I. L; Muraveiskaya; G. S.; Korablina, L. S. Russ. J. Inorg. Chem. 1965, 10, 158; (b) Chemyaev, Muraveiskaya, G. S.; Korablina, L. S. Russ. J. Inorg. Chem. 1996, 11, 728; and (c) Cehmayev, 1. I; Leonova, T. N. Russ. J. Inorg. Chem. 1969, 14, 307. More recently, a number of publications describe the characteristics of these and similar compounds (for example, Turkon, J.; Zhang, S.; Palmer, J. Kay, H., Stanko, J. Mora, L. B. Sebti, S., Yu, H. Jove, R. Molecular Cancer Therapeutics, 2004, 3, 1533, US 2005/0288365, US 2005/0080131, US 2007/0161613 and U.S. Pat. No. 5,849,790. However, there is still a need to develop novel platinum complexes, such as nitroplatinum(IV) complexes, which can be readily synthesized and provide anticancer activity.

The disclosures of all publications, patents, patent applications and other references referred to herein are hereby incorporated herein by reference in their entireties.

### BRIEF SUMMARY OF THE INVENTION

One aspect described herein provides a platinum(IV) complex comprising a nitro ligand and a bidentate dicarboxylate ligand.

In another aspect, is provided a platinum complex of formula (I) or (II):

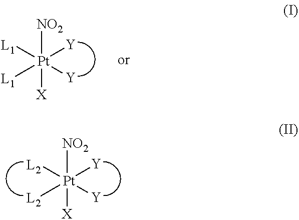

wherein each L₁ is independently a monodentate nitrogen donor ligand; L₂-L₂ is a bidentate ligand which forms a 5-8 membered chelate ring with the platinum atom and wherein the donor atoms of the bidentate ligand are each independently N or S; Y—Y is a dicarboxylate linked to the platinum atom through the terminal oxygen atoms; and X is either a halide, —ONO₂, or a carboxylate linked through its oxygen atom; or a pharmaceutically acceptable salt thereof or solvate of the foregoing.

In some embodiments of the complex of formula (I) or (II), the dicarboxylate is a C₂-C₇ dicarboxylate. In some embodiments, the dicarboxylate is an oxalate. In some embodiments, X is a halide.

In some embodiments of the complex of formula (I), each L₁ is the same. In some embodiments, each L₁ is different. In some embodiments, at least one L₁ is NH₃.

In some embodiments of the complex of formula (II), the donor atoms of the bidentate ligand L₂-L₂ are both N. In some embodiments, one donor atom of the bidentate ligand L₂-L₂ is N and the other donor atom is S. In some embodiments, at least one N donor atom is aromatic (e.g., imidazole). In some embodiments, the bidentate ligand L₂-L₂ forms a 5 or 6-membered chelate ring with the platinum atom. In some embodiments, the bidentate ligand L₂-L₂ comprises cycloalkyl or heterocycloalkyl. In some embodiments, the bidentate ligand L₂-L₂ comprises aryl or heteroaryl.

In some embodiments, the platinum complex is cis-diammineoxalatochloronitroplatinum(IV); cis-diammineoxalatobromonitroplatinum(IV); cis-diammine(cyclobutane-1,1-dicarboxylato)chloronitroplatinum(IV); cis-diammine(cyclobutane-1,1-dicarboxylato)bromonitroplatinum(IV); (trans-l-1,2-diaminocyclohexane)oxalatochloronitro-platinum(IV); (trans-l-1,2-diaminocyclohexane)oxalatobromonitro-platinum(IV); (1-butyl-2-(aminomethyl)imidazole)oxalatochloronitroplatinum(IV); (2-amino-3-(4-imidazolyl)propionate)oxalato-chloronitroplatinum(IV); (2-amino-3-(4-imidazolyl)methylpropionate) oxalato-chloronitroplatinum(IV); (1-methyl-2-(aminophenylmethyl)imidazole)oxalato-chloronitroplatinum(IV); (1-butyl-2-(methylthiomethyl)imidazole)oxalatochloro-nitroplatinum(IV); (1-methyl-2-(methylthiomethyl)imidazole) oxalatochloro-nitroplatinum(IV); or a pharmaceutically acceptable salt thereof or solvate of the foregoing.

In some embodiments of formula (I) or (II), while not being bound to any theory, it is possible that the complex has increased stability relative to the corresponding platinum(II) complex lacking X and NO₂.

In another aspect, is provided a formulation comprising a platinum complex of formula (I) or (II), or a pharmaceutically acceptable salt thereof or solvate of the foregoing, and a carrier. In some embodiments, the carrier is a pharmaceutically acceptable carrier. In some embodiments, the formulation comprises an effective amount of a platinum complex of formula (I) or (II), or a pharmaceutically acceptable salt thereof or solvate of the foregoing, and a carrier (e.g., a pharmaceutically acceptable carrier). In some embodiments, the platinum complex of formula (I) or (II) are of substantially pure form.

In another aspect, are provided methods of treating a proliferative disease in an individual (e.g., cancer), comprising administering to the individual an effective amount of a platinum complex of formula (I) or (II). In some embodiments, the cancer is a solid tumor. In some embodiments, the cancer is selected from the group consisting of colorectal cancer, multiple myeloma, renal cell carcinoma, prostate cancer, lung cancer, melanoma, ovarian cancer, and breast cancer. In some embodiments, the platinum complex is administered parenterally. In some embodiments, the platinum complex is administered enterally (e.g., orally).

In another aspect, are provided methods of inhibiting and/or delaying proliferation of cells, comprising contacting the cells with a platinum complex of formula (I) or (II).

In another aspect, are provided methods for the preparation of a platinum complex of formula (I) or (II), comprising reacting a platinum(II) complex in a suitable solvent with a halide in anionic form or carboxylate in anionic form, and NO₂.

### BRIEF DESCRIPTION OF THE FIGURES

FIG. 1 shows the crystallographic structure of complex II-A (trans-l-1,2-diaminocyclohexane)oxalatochloronitro-platinum(IV)).

FIG. 2 shows the positive ion mass spectra comparing complex II-A (depicted as Mar4.1.4): (trans-l-1,2-diaminocyclohexane)oxalatochloronitro-platinum(IV) (water/37° C./18 h) in the absence (top) and presence (bottom) of cysteine.

### DETAILED DESCRIPTION OF THE INVENTION

Provided herein are nitroplatinum(IV) complexes comprising a bidentate dicarboxylate ligand (e.g., oxalate). Strikingly, despite oxalate being a known reducing agent and likely to become oxidized by NO₂, certain platinum(IV) compounds containing oxalate and NO₂ are readily preparable and relatively stable. Such complexes may be useful for cancer treatment and may provide increased cytotoxic selectivity of cancerous cells, reduced toxicity, and/or increased relative water solubility. These complexes may be particularly useful for enterally administered cancer treatment.

In one aspect, are provided platinum(IV) complexes as described herein. In another aspect, are provided methods of treating cancer using the platinum(IV) complexes described herein. Also provided are kits and unit dosage forms of the platinum (IV) complexes.

### ABBREVIATIONS AND DEFINITIONS

The terms “halo” or “halogen,” by themselves or as part of another substituent, mean, unless otherwise stated, a fluorine, chlorine, bromine, or iodine atom.

The term “alkyl,” by itself or as part of another substituent, means, unless otherwise stated, a fully saturated straight-chain (linear; unbranched) or branched chain, or combination thereof, having the number of carbon atoms specified, if designated (i.e. C₁-C₁₀ means one to ten carbons). Examples include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like. If no size is designated, the alkyl groups mentioned herein contain 1-20 carbon atoms, typically 1-10 carbon atoms, or 1-8 carbon atoms, or 1-6 carbon atoms, or 1-4 carbon atoms. The term “alkylene” is by itself or in combination with other terms, represents a divalent radical derived from an alkyl, as exemplified, but not limited, by —CH₂CH₂CH₂CH₂—. In some embodiments, the alkylene group is methylene or ethylene.

The term “alkenyl” refers to unsaturated aliphatic groups including straight-chain (linear; unbranched), branched-chain groups, and combinations thereof, having the number of carbon atoms specified, if designated, which contain at least one double bond (—C═C—). All double bonds may be independently either (E) or (Z) geometry, as well as mixtures thereof. Examples of alkenyl groups include, but are not limited to, —CH₂—CH═CH—CH₃; —CH═CH—CH═CH₂ and —CH₂—CH═CH—CH(CH₃)—CH₂—CH₃. If no size is designated, the alkenyl groups mentioned herein contain 2-20 carbon atoms, typically 2-10 carbon atoms, or 2-8 carbon atoms, or 2-6 carbon atoms, or 2-4 carbon atoms. Alkenyl may be substituted or unsubstituted unless otherwise noted.

The term “alkynyl” refers to unsaturated aliphatic groups including straight-chain (linear; unbranched), branched-chain groups, and combinations thereof, having the number of carbon atoms specified, if designated, which contain at least one carbon-carbon triple bond (—C≡C—). Examples of alkynyl groups include, but are not limited to, —CH₂—C≡C—CH₃; —C≡C—C≡CH and —CH₂—C≡C—CH(CH₃)—CH₂—CH₃. If no size is designated, the alkynyl groups mentioned herein contain 2-20 carbon atoms, typically 2-10 carbon atoms, or 2-8 carbon atoms, or 2-6 carbon atoms, or 2-4 carbon atoms.

The term “cycloalkyl” by itself or in combination with other terms, represents, unless otherwise stated, cyclic versions of alkyl, alkenyl, or alkynyl, or mixtures thereof. Additionally, cycloalkyl may contain fused rings, but excludes fused aryl and heteroaryl groups. Examples of cycloalkyl include, but are not limited to, cyclopropyl, cyclobutyl, cyclopentyl, cyclohexyl, 1-cyclohexenyl, 3-cyclohexenyl, cycloheptyl, norbornyl, and the like. If no size is designated, the alkynyl groups mentioned herein contain 3-9 carbon atoms, typically 3-7 carbon atoms.

The term “heterocycloalkyl,” by itself or in combination with other terms, represents a saturated or unsaturated cyclic hydrocarbon radical containing of at least one carbon atom and at least one annular heteroatom selected from the group consisting of O, N, P, Si and S, and wherein the nitrogen and sulfur atoms may optionally be oxidized and the nitrogen heteroatom may optionally be quaternized. The heteroatom(s) O, N, P, S and Si may be placed at any interior position of the heterocycloalkyl group or at the position at which the heterocycloalkyl group is attached to the remainder of the molecule. Examples of heterocycloalkyl include, but are not limited to, thiazolidinonyl, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “cycloalkyl-alkyl” and “heterocycloalkyl-alkyl” designate an alkyl-substituted cycloalkyl group and alkyl-substituted heterocycloalkyl, respectively, where the alkyl moiety is attached to the parent structure. Non-limiting examples include cyclopropyl-ethyl, cyclobutyl-propyl, cyclopentyl-hexyl, cyclohexyl-isopropyl, 1-cyclohexenyl-propyl, 3-cyclohexenyl-t-butyl, cycloheptyl-heptyl, norbornyl-methyl, 1-piperidinyl-ethyl, 4-morpholinyl-propyl, 3-morpholinyl-t-butyl, tetrahydrofuran-2-yl-hexyl, tetrahydrofuran-3-yl-isopropyl, and the like. Cycloalkyl-alkyl and heterocycloalkyl-alkyl also include substituents in which at least one carbon atom is present in the alkyl group and wherein another carbon atom of the alkyl group has been replaced by, for example, an oxygen, nitrogen or sulfur atom (e.g., cyclopropoxymethyl, 2-piperidinyloxy-t-butyl, and the like).

The term “aryl” means, unless otherwise stated, a polyunsaturated, aromatic, hydrocarbon substituent which can be a single ring or multiple rings (e.g., from 1 to 3 rings) which are fused together or linked covalently. Additionally, aryl may contain fused rings, wherein one or more of the rings are optionally cycloalkyl or heterocycloalkyl. Examples of aryl groups include, but are not limited to, phenyl, 1-naphthyl, 2-naphthyl, 4-biphenyl.

The term “heteroaryl” refers to aryl groups (or rings) that contain from one to four annular heteroatoms selected from N, O, and S, wherein the nitrogen and sulfur atoms are optionally oxidized, and the nitrogen atom(s) are optionally quaternized. A heteroaryl group can be attached to the remainder of the molecule at an annular carbon or annular heteroatom. Additionally, heteroaryl may contain fused rings, wherein one or more of the rings are optionally cycloalkyl or heterocycloalkyl. Non-limiting examples of heteroaryl groups are 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

The term “aralkyl” designates an alkyl-substituted aryl group, where the alkyl portion is attached to the parent structure. Examples are benzyl, phenethyl, and the like. “Heteroaralkyl” designates a heteroaryl moiety attached to the parent structure via an alkyl residue. Examples include furanylmethyl, pyridinylmethyl, pyrimidinylethyl, and the like. Aralkyl and heteroaralkyl also include substituents in which at least one carbon atom of the alkyl group is present in the alkyl group and wherein another carbon of the alkyl group has been replaced by, for example, an oxygen atom (e.g., phenoxymethyl, 2-pyridylmethoxy, 3-(1-naphthyloxy)propyl, and the like).

Each of the above terms (e.g., “alkyl,” “alkenyl,” “alkynyl,” “cycloalkyl,” “heterocycloalkyl,” “cycloalkyl-alkyl,” “heterocycloalkyl-alkyl,” “aryl,” “heteroaryl,” “aralkyl,” and “heteroaralkyl”) are meant to include, unless otherwise indicated, both substituted and unsubstituted forms of the stated radical.

“Optionally substituted” or “substituted” refers to the replacement of one or more hydrogen atoms with a monovalent or divalent radical. Suitable substituent groups include, for example, hydroxyl, nitro, amino, imino, cyano, halo (such as F, Cl, Br, I), haloalkyl (such as —CCl₃ or —CF₃), thio, sulfonyl, thioamido, amidino, imidino, oxo, oxamidino, methoxamidino, imidino, guanidino, sulfonamido, carboxyl, formyl, alkyl, alkoxy, alkoxy-alkyl, alkylcarbonyl, alkylcarbonyloxy (—OCOR), aminocarbonyl, arylcarbonyl, aralkylcarbonyl, carbonylamino, heteroarylcarbonyl, heteroaralkyl-carbonyl, alkylthio, aminoalkyl, cyanoalkyl, carbamoyl (—NHCOOR— or —OCONHR—), urea (—NHCONHR—), aryl and the like. In some embodiments, the above groups (e.g., alkyl groups) are substituted with, for example, amino, heterocycloalkyl, such as morpholine, piperazine, piperidine, azetidine, hydroxyl, methoxy, or heteroaryl groups such as pyrrolidine.

A substituent group can itself be substituted. The group substituted onto the substitution group can be, for example, carboxyl, halo, nitro, amino, cyano, hydroxyl, alkyl, alkenyl, alkynyl, alkoxy, aminocarbonyl, —SR, thioamido, —SO₃H, —SO₂R or cycloalkyl, where R is e.g., a hydrogen or alkyl.

When the substituted substituent includes a straight chain group, the substituent can occur either within the chain (e.g., 2-hydroxypropyl, 2-aminobutyl, and the like) or at the chain terminus (e.g., 2-hydroxyethyl, 3-cyanopropyl, and the like). Substituted substituents can be straight chain, branched or cyclic arrangements of covalently bonded carbon or heteroatoms (N, O or S).

A “monodentate ligand” or variant thereof refers to a ligand which binds the platinum metal at one site. A “bidentate ligand” or variant thereof refers to a ligand which binds the platinum metal at two sites. It is understood that as used herein, a monodentate or bidentate ligand may contain additional atoms capable of binding the platinum metal, but wherein the ligand binds the metal with only the indicated number of sites. For example, a ligand containing two nitrogens would be considered a monodentate ligand if only one nitrogen binds the platinum metal, and would be considered a bidentate ligand if both nitrogens bind the platinum metal.

As used herein, “treatment”, “treating”, or “treat” is an approach for obtaining beneficial or desired results, including clinical results. For purposes herein, beneficial or desired results include, but are not limited to, one or more of the following: decreasing one more symptoms resulting from the disease (e.g., cancer), diminishing the extent of the disease, stabilizing the disease (e.g., preventing or delaying the worsening of the disease, such as cancer), delay or slowing the progression of the disease, ameliorating the disease state, decreasing the dose of one or more other medications required to treat the disease, increasing the quality of life of an individual who has been or is suspected of having the disease, and/or prolonging survival (including overall survival and progression free survival). Also encompassed by “treatment” is a reduction of pathological consequence of cancer. The methods described herein contemplate any one or more of these aspects of treatment.

As used herein, “delaying” means to defer, hinder, slow, retard, stabilize, and/or postpone development of, and/or one or more symptoms of the disease (e.g., cancer). This delay can be of varying lengths of time, depending on the history of the disease and/or individual being treated. As is evident to one skilled in the art, a sufficient or significant delay can, in effect, encompass prevention, in that the individual does not develop the disease (e.g., cancer). A method that “delays” development of cancer is a method that reduces the probability of disease development in a given time frame and/or reduces the extent of the disease in a given time frame, when compared to not using the method. Such comparisons are typically based on clinical studies, using a statistically significant number of subjects. Cancer development can be detectable using standard methods, such as routine physical exams or x-ray. Development may also refer to disease progression that may be initially undetectable and includes occurrence and onset.

As used herein, an “at risk” individual is an individual who is at risk of developing a disease (e.g., cancer). An individual “at risk” may or may not have a detectable disease, and may or may not have displayed symptoms associated with a detectable disease prior to the treatment methods described herein. “At risk” denotes that an individual has one or more so-called risk factors, which are measurable parameters that correlate with development of the disease. An individual having one or more of these risk factors has a higher probability of developing the disease than an individual without these risk factor(s).

As used herein, “pharmaceutically acceptable” refers to a material that is not biologically or otherwise undesirable, e.g., the material may be incorporated (e.g., at the time of manufacturing or administration) into a pharmaceutical composition administered to an individual without causing any significant undesirable biological effects or interacting in a deleterious manner with any of the other components of the composition in which it is contained. As used herein, the term “pharmaceutically acceptable carrier,” refers to, for example, solvents, stabilizers, pH-modifiers, tonicity modifiers, adjuvants, binders, diluents, etc., known to the skilled artisan that are suitable for administration to an individual (e.g., a human). Combinations of two or more carriers are also contemplated. The pharmaceutically acceptable carrier(s) and any additional components, as described herein, should be compatible for use in the intended route of administration (e.g., oral, parenteral) for a particular dosage form. Such suitability will be easily recognized by the skilled artisan, particularly in view of the teaching provided herein. Pharmaceutically acceptable carriers or excipients have preferably met the required standards of toxicological and manufacturing testing and/or are included on the Inactive Ingredient Guide prepared by the U.S. Food and Drug administration.

An “effective amount,” as used herein refer to an amount that results in a desired pharmacological and/or physiological effect for a specified disease (e.g., cancer) or one or more of its symptoms and/or to completely or partially prevent the occurrence or recurrence of the disease or symptom thereof and/or may be therapeutic in terms of a partial or complete cure for the condition and/or adverse effect attributable to the condition (e.g., cancer). In reference to conditions described herein (e.g., cancer), a pharmaceutically or therapeutically effective amount may comprise an amount sufficient to, among other things, reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; prevent growth and/or kill existing cancer cells; be cytostatic and/or cytotoxic; restore or maintain vasculostasis or prevention of the compromise or loss or vasculostasis; reduction of tumor burden; reduction of morbidity and/or mortality; and/or relieve to some extent one or more of the symptoms associated with the cancer. The effective amount may extend progression free survival (e.g. as measured by Response Evaluation Criteria for Solid Tumors, RECIST, or CA-125 changes), result in an objective response (including a partial response or a complete response), increase overall survival time, and/or improve one or more symptoms of cancer (e.g. as assessed by FOSI). In certain embodiments, the pharmaceutically effective amount is sufficient to prevent the condition, as in being administered to an individual prophylactically. Effective amount includes the eradication or amelioration of the underlying condition being treated and/or eradication or amelioration of one or more of the symptoms associated with the underlying condition such that the individual reports an improvement in feeling or condition (e.g., decreased pain intensity and/or duration), notwithstanding that the individual may still be afflicted with the underlying disease. Effective amount also includes halting or slowing the progression of the disease (e.g., cancer), regardless of whether improvement or the disease or condition is realized.

The “effective amount” may vary depending on the composition being administered, the condition being treated/prevented (e.g., the type of cancer), the severity of the condition being treated or prevented, the age, body size, weight, and relative health of the individual, the route and form of administration, the judgment of the attending medical or veterinary practitioner (if applicable), and other factors appreciated by the skilled artisan in view of the teaching provided herein. An effective amount may be assessed, for example, by using data from one or more clinical, physiological, biochemical, histological, electrophysiological, and/or behavioral evaluations.

As is understood in the art, an “effective amount” may be in one or more doses, i.e., a single dose or multiple doses may be required to achieve the desired treatment endpoint. An effective amount may be considered in the context of administering one or more additional pharmaceutical agents, and a platinum(IV) complex may be considered to be given in an effective amount if, in conjunction with one or more additional pharmaceutical agents, one or more desirable or beneficial result(s) may be or are achieved.

When used with respect to methods of treatment/prevention and the use of the platinum(IV) complexes and compositions thereof described herein, an individual “in need thereof” may be an individual who has been diagnosed with, previously treated for, and/or suspected of having the disease to be treated (e.g., a proliferative disease such as cancer). With respect to prevention, the individual in need thereof may also be an individual who is at risk for a condition (e.g., a family history of the condition, life-style factors indicative of risk for the condition, etc.).

In some embodiments, the individual is a mammal, including, but not limited to, bovine, horse, feline, rabbit, canine, rodent, or primate. In some embodiments, the mammal is a primate. In some embodiments, the primate is a human. In some embodiments, the individual is human, including adults, children, infants, and preemies. In some embodiments, the individual is a non-mammal. In some variations, the primate is a non-human primate such as chimpanzees and other apes and monkey species. In some embodiments, the mammal is a farm animal such as cattle, horses, sheep, goats, and swine; pets such as rabbits, dogs, and cats; laboratory animals including rodents, such as rats, mice, and guinea pigs; and the like. In some embodiments, the individual is a non-mammal, including, but not limited to, birds, and the like. The term “individual” does not denote a particular age or sex.

As used herein, “combination therapy” means a first therapy that includes a platinum(IV) complex in conjunction with a second therapy (e.g., surgery and/or an additional pharmaceutical agent) useful for treating, stabilizing, preventing, and/or delaying the disease or condition. Administration in “conjunction with” another compound includes administration in the same or different composition(s), either sequentially, simultaneously, or continuously, through the same or different routes. In some embodiments, the combination therapy optionally includes one or more pharmaceutically acceptable carriers or excipients, non-pharmaceutically active compounds, and/or inert substances.

As used herein, the term “additional pharmaceutical agent,” refers to an active agent other than a platinum(IV) complex, for example, a drug, which is administered to elicit a therapeutic effect. The pharmaceutical agent(s) may be directed to a therapeutic effect related to the condition that platinum(IV) complexes are intended to treat or prevent (e.g., cancer) or, the pharmaceutical agent may be intended to treat or prevent a symptom of the underlying condition (e.g., tumor growth, hemorrhage, ulceration, pain, enlarged lymph nodes, cough, jaundice, swelling, weight loss, cachexia, sweating, anemia, paraneoplastic phenomena, thrombosis, etc.) or to further reduce the appearance or severity of side effects of the platinum(IV) complexes.

Reference to “about” a value or parameter herein includes (and describes) variations that are directed to that value or parameter per se. For example, a description referring to “about X” includes the description of “X”.

As used herein and in the appended claims, the singular forms “a,” “or,” and “the” include plural referents unless the context clearly dictates otherwise. It is understood that aspect and variations described herein include “consisting” and/or “consisting essentially of” aspects and variations.

Unless defined otherwise or clearly indicated by context, all technical and scientific terms and abbreviations used herein have the same meaning as commonly understood by one of ordinary skill in the art to which this invention belongs.

### Platinum(IV) Complexes

Described herein are platinum(IV) complexes which may be useful in the treatment of diseases (e.g., proliferative diseases, such as cancer). The complexes contain nitroplatinum(IV) and a bidentate dicarboxylate coligand (e.g., oxalate).

In one aspect, the platinum complex is of the formula (I) or (II):

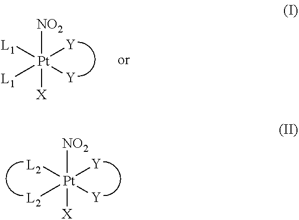

wherein each L₁ is independently a monodentate nitrogen donor ligand; L₂-L₂ is a bidentate ligand which forms a 5-8 membered dictate ring with the platinum atom and wherein the donor atoms of the bidentate ligand are each independently N or S; Y—Y is a dicarboxylate linked to the platinum atom through the terminal oxygen atoms; and X is either a halide, or a carboxylate linked through the oxygen atom; or a pharmaceutically acceptable salt thereof or solvate of the foregoing. In some embodiments, the dicarboxylate is a C₂-C₇ dicarboxylate. In some embodiments, the dicarboxylate is a C₂-C₃ dicarboxylate. In some embodiments, the dicarboxylate is cyclobutane-1,1-dicarboxylate. In some embodiments, the dicarboxylate is an oxalate.

In some embodiments, the platinum complex is of formula (III) or (IV):

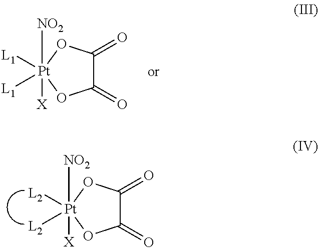

wherein each L₁ is independently a monodentate nitrogen donor ligand; L₂-L₂ is a bidentate ligand which forms a 5-8 membered chelate ring with the platinum atom and wherein the donor atoms of the bidentate ligand are each independently N or S; and X is a halide, —ONO₂, or a carboxylate linked through the oxygen atom; or a pharmaceutically acceptable salt thereof or solvate of the foregoing.

In some embodiments of formula (I) or (III), each L₁ is independently an N-containing heteroaryl, —NH₃ or —NHR¹; wherein each R¹ is independently a substituted or unsubstituted moiety selected from alkyl, alkenyl, alkynyl, cycloalkyl, cycloalkyl-alkyl, aryl, aralkyl, heteroaryl, and heteroaralkyl. In some embodiments, each L₁ is the same. In some embodiments, each L₁ is different. In some embodiments, at least one L₁ is —NH₃. In some embodiments, each L₁ is —NH₃.

In some embodiments of formula (II) or (IV), the donor atoms of the bidentate ligand L₂-L₂ are both N. In some embodiments, one donor atom of the bidentate ligand L₂-L₂ is N and the other donor atom is S. In some embodiments, L₂-L₂ comprises at least one aromatic or non-aromatic cyclic N donor atom (e.g., an N-containing heteroaryl, such as pyridine or imidazole, or an N-containing heterocycle, such as piperidine or piperazine). In some embodiments, L₂-L₂ comprises at least one aliphatic N donor atom. In some embodiments, L₂-L₂ comprises at least one N donor atom which is an exocyclic amine (e.g., from an aromatic or non-aromatic 5-8 membered ring). In some embodiments, both N donor atoms of L₂-L₂ are exocyclic amines (e.g., from an aromatic or non-aromatic 5-8 membered ring). In some embodiments, L₂-L₂ forms a 5 or 6-membered chelate ring with the platinum atom. In some embodiments, L₂-L₂ forms a 5-membered chelate ring with the platinum atom. In some embodiments, L₂-L₂ forms a 6-membered chelate ring with the platinum atom. In some embodiments, the bidentate ligand L₂-L₂ comprises cycloalkyl or heterocycloalkyl. In some embodiments, the bidentate ligand L₂-L₂ comprises cycloalkyl. In some embodiments, the cycloalkyl is a 5-8 membered cycloalkyl. In some embodiments, the cycloalkyl is cyclohexane. In some embodiments, L₂-L₂ comprises aryl or heteroaryl. In some embodiments, L₂-L₂ comprises a heteroaryl (e.g., imidazole, pyridine, pyrazine, or pyrazine).

Examples of neutral bidentate ligands (L₂-L₂) which comprise two N donor atoms include: (i) diaminocyclohexane; (ii) 1-butyl-2-(aminomethyl)imidazole; (iii) 1-methyl-2-(aminophenylmethyl)imidazole; (iv) 2-amino-3-(4-imidazolyl)methylpropionate; and (v) 2-amino-3-(4-imidazolyl)propionic methyl ester.

Examples of neutral bidentate ligands (L₂-L₂) which comprise an N donor atom and a donor atom other than N (e.g., thioethereal groups, such as those found in 1-alkyl/aryl-2-alkylthioalkyl/aryl heterocyclic amines, particularly imidazoles) include: (vi) 1-methyl-2-(methylthiomethyl)imidazole; (vii) 1-butyl-2-(methylthiomethyl)imidazole; (viii) 1-methyl-2-(methylthioethyl)imidazole; (ix) 1-methyl-2-(methylthiopropyl)imidazole; (x) 1-butyl-2-(methylthioethyl)imidazole; (xi) 2-(methylthiomethyl)pyridine; (xii) 2-(methylthioethyl)pyridine; (xiii) 2-(methylthiopropyl)pyridine; (xiv) 1-amino-2-thiomethyl-ethane; (xv) 1-amino-2-thioethyl-ethane; (xvi) 2,5-dithiahexane; and (xvii) 2,5-diselenohexane.

In some embodiments of any one of formulas (I), (II), (III) or (IV), X is a halide. In some embodiments, X is Cl or Br. In some embodiments, X is Br. In some embodiments, X is Cl. In some embodiments, X is a carboxylate linked through the oxygen atom. In some embodiments, X is —OC(O)R²; wherein R² is independently a substituted or unsubstituted moiety selected from alkyl, alkenyl, alkynyl, cycloalkyl, cycloalkyl-alkyl, aryl, aralkyl, heteroaryl, and heteroaralkyl. In some embodiments, R² is a substituted or unsubstituted alkyl or a substituted or unsubstituted aryl. In some embodiments, X is —ONO₂.

In some embodiments, the complex of formula (I) is the compound:

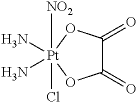

(I-A): cis-diammineoxalatochloronitroplatinum(IV);

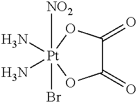

(I-B): cis-diammineoxalatobromonitroplatinum(IV);

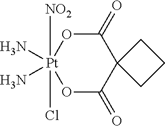

(I-C): cis-diammine(cyclobutane-1,1-dicarboxylato)chloronitroplatinum(IV);

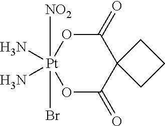

(I-D): cis-diammine(cyclobutane-1,1-dicarboxylato)bromonitroplatinum(IV);

or a pharmaceutically acceptable salt thereof or solvate of the foregoing.

In some embodiments, the complex of formula (II) is the compound:

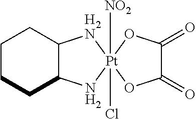

(II-A): (trans-l-1,2-diaminocyclohexane)oxalatochloronitro-platinum(IV);

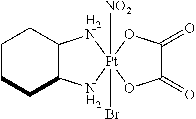

(II-B): (trans-l-1,2-diaminocyclohexane)oxalatobromonitro-platinum(IV);

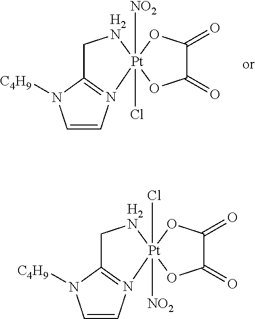

(II-C): (1-butyl-2-(aminomethyl)imidazole)oxalatochloronitroplatinum(IV);

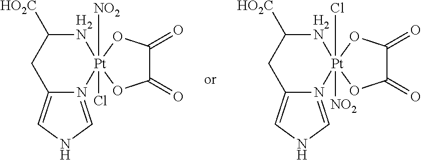

(II-D): (2-amino-3-(4-imidazolyl)propionate)oxalato-chloronitroplatinum(IV);

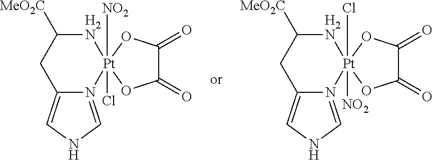

(II-E): (2-amino-3-(4-imidazolyl)methylpropionate)oxalato-chloronitroplatinum(IV);

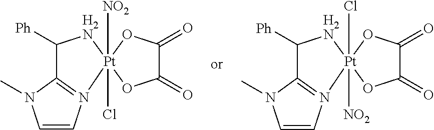

(II-F): (1-methyl-2-(aminophenylmethyl)imidazole)oxalato-chloronitroplatinum(IV);

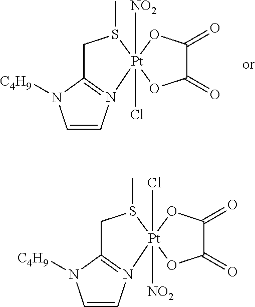

(II-G): (1-butyl-2-(methylthiomethyl)imidazole)oxalatochloro-nitroplatinum(IV);

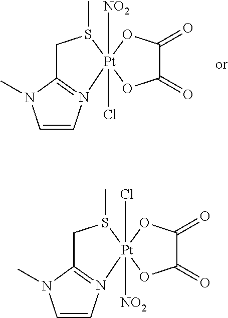

(II-H): (1-methyl-2-(methylthiomethyl)imidazole) oxalatochloro-nitroplatinum(IV);

or a pharmaceutically acceptable salt thereof or solvate of the foregoing.

In some embodiments, the platinum(IV) complex (e.g., any complex of formula I, II, III, or IV) is in substantially pure form. Unless otherwise stated, “substantially pure” intends a preparation of the platinum(IV) complex that contains no more than 15% impurity, wherein the impurity intends compounds other than the platinum(IV) complex, but does not include other forms of the complex (e.g., different salt form or a different stereoisomer, conformer, rotamer, or tautomer of the platinum (IV) complexes depicted). In one variation, a preparation of substantially pure platinum (IV) complex is provided wherein the preparation contains no more than 25% impurity, or no more than 20% impurity, or no more than 10% impurity, or no more than 5% impurity, or no more than 3% impurity, or no more than 1% impurity, or no more than 0.5% impurity. In some embodiments, the platinum(IV) complex (e.g., any complex of formula I, II, III, or IV) does not contain trace amounts of silver. Methods of making platinum(II) complexes without the use of silver are described in US 2007/0167643, US 2008/0064895, and WO 2007/085957 (the contents of which are incorporated herein by reference in their entireties). In some embodiments, the corresponding platinum(IV) complexes to the platinum(II) complexes described in these applications are contemplated. For example, it is contemplated that complexes (i) through (viii) of US 2007/0167643 may be converted into their corresponding platinum(IV) complexes by the addition of NO₂ and Cl or Br ligands, as taught and described herein.

The platinum(IV) complexes described herein and methods of using the same include all solvate and/or hydrate forms. In some embodiments, the platinum(IV) complexes described herein can exist in unsolvated forms as well as solvated forms (i.e., solvates). The platinum(IV) complexes may also include hydrated forms (i.e., hydrates).

The platinum(IV) complexes described herein (e.g., any complex of formula I, II, III, or IV), as well as methods of using such salts of the complexes, include all salt forms of the complexes. The platinum(IV) complexes also include all non-salt forms of any salt of a platinum(IV) complex described herein, as well as other salts of any salt of a platinum(IV) complex named herein. In some embodiments, the salts of the platinum (IV) complex are pharmaceutically acceptable salts. “Pharmaceutically acceptable salts” are those salts which retain the biological activity of the free prodrugs and which can be administered as drugs or pharmaceuticals to and individual (e.g., a human). The desired salt of a basic functional group of a compound may be prepared by methods known to those of skill in the art by treating the compound with an acid. The desired salt of an acidic functional group of a compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, bismuth salts, and calcium salts; ammonium salts; and aluminum salts. Examples of organic salts of acid compounds include, but are not limited to, procaine, dibenzylamine, N-ethylpiperidine, N,N′-dibenzylethylenediamine, trimethylamine, and triethylamine salts. Examples of inorganic salts of base compounds include, but are not limited to, hydrochloride and hydrobromide salts. Examples of organic salts of base compounds include, but are not limited to, tartrate, citrate, maleate, fumarate, and succinate.

Unless stereochemistry is explicitly indicated in a chemical structure or chemical name, the chemical structure or chemical name is intended to embrace all possible stereoisomers, conformers, rotamers, and tautomers of the platinum(IV) complexes depicted. For example, a complex containing a chiral carbon atom is intended to embrace both the (R) enantiomer and the (S) enantiomer, as well as mixtures of enantiomers, including racemic mixtures; and a compound containing two chiral carbons is intended to embrace all enantiomers and diastereomers (including (R,R), (S,S), (R,S), and (R,S) isomers).

Included in all uses of the complexes of the formulas disclosed herein, is any or all of the stereochemical, enantiomeric, diastereomeric, conformational, rotomeric, tautomeric, solvate, hydrate, salt, and pharmaceutically acceptable salt of the complexes as described.

In some embodiments, without being bound to any theory, certain platinum (IV) complexes may be prepared apparently through the stabilizing effect of the highly covalently bonded nitro group and other coligands. In some embodiments, and without being bound to any theory, the oxalato group forming a five-membered chelate bond to the platinum(II) results in a relatively kinetically stable complex, which may facilitate the synthetic process resulting in relatively high yields of the final product in high degrees of purity. It is possible that the amine ligands and a nitroligand may aid in stabilizing these platinum(IV) compounds, even in some cases of N—S chelate ligands. Accordingly, in some embodiments, the platinum complex (e.g., a complex of any one of formulas (I)-(IV)) comprises ligands L₁ or L₂-L₂ which result in the complex having an increased stability relative to the corresponding platinum(II) complex lacking NO₂ and X. Increased stability may include increased structural integrity of the platinum(IV) complex under various environmental conditions in vivo and/or ex vivo.

Some platinum(IV) complexes described herein may have improved reactivity profiles useful for the treatment of certain types of diseases (e.g., cancer) when compared to certain platinum(II) compounds (e.g., cisplatin and oxaliplatin). For example, certain platinum(IV) complexes described herein may be particularly reactive toward cancerous cells when compared to non-cancerous cells, resulting in improved selectivity during treatment. Without being bound by theory, some platinum(IV) complexes described herein may be relatively less reactive toward biological nucleophiles in vivo. Such complexes may convert into a more reactive form, such as a corresponding platinum(II) complex (e.g., a complex lacking NO₂ and X), upon exposure to the reducing environment found within cancerous cells, and thus potentially increasing selective cyclotoxicity and reducing the overall toxicity profile. Example 8 demonstrates how a platinum(IV) complex may be converted into its corresponding platinum(II) complex under simulated in vivo conditions. In some embodiments, a platinum(IV) complex (e.g., a complex of any one of formulas (I)-(IV)) is less reactive (e.g., toward non-cancerous cells) then its corresponding platinum(II) complex lacking NO₂ and X. In some embodiments, the platinum(IV) complex (e.g., a complex of any one of formulas (I)-(IV)) is converted, or is capable of being converted (e.g., in vivo and/or under physiological conditions) into its corresponding platinum(II) complex lacking NO₂ and X. In some embodiments, the platinum(IV) complex is more cytotoxic after being converted (e.g., in vivo and/or under physiological conditions) into its corresponding platinum(II) complex lacking NO₂ and X. In some embodiments, the platinum(IV) complex itself is cytotoxic. In some embodiments, the platinum(IV) complex is not significantly converted or is not capable of being significantly converted (e.g., in vivo and/or under physiological conditions) into its corresponding platinum(II) complex lacking NO₂ and X.

The platinum(IV) complexes described herein may have improved water solubility relative to existing platinum complexes. The dicarboxylate ligands (e.g., oxalate) have polar non-coordinated oxygen atoms which may render these complexes relatively water soluble when compared to highly covalent chloro complexes of other, relatively insoluble, platinum(IV) complexes (e.g., Pt(NH₃)₂Cl₄). For example, (trans-l-1,2-diaminocyclohexane) oxalatobromonitro-platinum(IV) (complex II-B), has a water solubility of greater than 20 mg/mL. Increased water solubility may result in the platinum(IV) complexes being more suitable for particular types of administration (e.g., parenteral administration) and may also permit a higher blood level concentration, if desired, and/or allow a lower dosage (and/or a lower dose volume in the case of parenteral formulation) to obtain a desired blood level concentration. Accordingly, in some embodiments, the platinum(IV) complexes (e.g., a complex of any one of formulas (I)-(IV)) have increased water solubility relative to Pt(NH₃)₂Cl₄. In some embodiments, the platinum(IV) complexes are greater than 2, 3, 5, 10, 15, 25, 50, 100, 200, 500 or 1000 times more soluble in water compared to Pt(NH₃)₂Cl₄ under the same conditions.

### Synthetic Methods

The platinum(IV)oxalato compounds described herein may be prepared by suspending and/or dissolving a platinum(II)oxalato complex in a suitable solvent, followed by nitration. Specific preparation protocols for several complexes are illustrated in the Examples section herein. Typically, one mole equivalent of NaX (where X is a halide such as Cl⁻ or Br⁻, typically Cl⁻, or a monodentate carbonate) is added and NO₂ gas is introduced via a NO₂ source. The NO₂ oxidizes the platinum(II) oxalato complex, usually via a blue-green platinum intermediate species, to a platinum(IV)chloronitro species without oxidizing the coordinated oxalato group. We note that the use of certain ligands (e.g., ligands viii-xvii) with this protocol did not result in high yields of the desired complexes.

In some embodiments, are provided methods of preparing a platinum(IV) complex (e.g., a complex of any one of formulas (I)-(IV)) comprising reacting a platinum(II) complex (e.g., a complex of any one of formulas (I)-(IV) wherein the complex is the corresponding platinum(II) complex lacking NO₂ and X) with NO₂ and a suitable form of X (e.g., a halide anion, a nitrate anion, or a carboxylate anion).

In some embodiments are provided methods for the preparation of a platinum(IV) complex of formula (I):

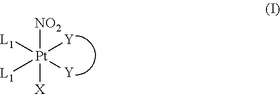

wherein L₁, Y—Y, and X are each as defined above; or a pharmaceutically acceptable salt thereof or solvate of the foregoing;
   comprising reacting a platinum(II) complex of formula (I-P):

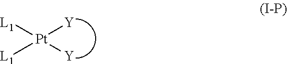

wherein each L₁ and Y—Y is as defined above;
   with NO₂ and a halide anion, nitrate anion, or carboxylate anion; in a suitable solvent to form a complex of formula (I), or a pharmaceutically acceptable salt thereof or solvate of the foregoing.

In some embodiments are provided methods for the preparation of a platinum(IV) complex of formula (II):

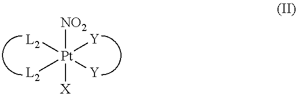

wherein L₂-L₂, Y—Y, and X are each as defined above; or a pharmaceutically acceptable salt thereof or solvate of the foregoing;
   comprising reacting a platinum(II) complex of formula (II-P):

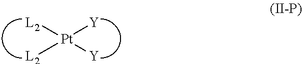

wherein L₂-L₂ and Y—Y is as defined above;
   with NO₂, and a halide, anion, or carboxylate anion; in a suitable solvent to form a complex of formula (II), or a pharmaceutically acceptable salt thereof or solvate of the foregoing.

In some embodiments of the methods for the preparation of a complex of formula (I) or (II) described above, the method further comprises recrystallization. In some embodiments, the halide in anionic form is generated in situ from a metal salt (e.g., a lithium, sodium, or potassium salt, such as in sodium chloride). In some embodiments, the suitable solvent is a polar solvent (e.g., acetone) and/or a protic solvent (e.g., water). In some embodiments, the suitable solvent is a mixed solvent (e.g., water:acetone). In some embodiments, the method does not oxidize the dicarboxylate (e.g., oxalate).

In some embodiments of the methods for the preparation of a complex of formula (I) or (II) described above, the platinum(IV) complex is produced in greater than about 50%, or about 60%, or about 70%, or about 80%, or about 85%, or about 90%, or about 95%, or about 97%, or about 98%, or about 99% yield.

In some embodiments of the methods for the preparation of a complex of formula (I) or (II) described above, the dicarboxylate Y—Y is a C₂-C₇ dicarboxylate. In some embodiments, the dicarboxylate is a C₂-C₃ dicarboxylate. In some embodiments, the dicarboxylate is cyclobutane-1,1-dicarboxylato. In some embodiments, the dicarboxylate is an oxalate. In some embodiments, X is a halide. In some embodiments, X is Cl or Br. In some embodiments, X is Br. In some embodiments, X is Cl. In some embodiments, X is a carboxylate linked through the oxygen atom. In some embodiments, X is —OC(O)R²; wherein R² is independently a substituted or unsubstituted moiety selected from alkyl, alkenyl, alkynyl, cycloalkyl, cycloalkyl-alkyl, aryl, aralkyl, heteroaryl, and heteroaralkyl. In some embodiments, R² is a substituted or unsubstituted alkyl or a substituted or unsubstituted aryl. In some embodiments, X is —ONO₂.

In some embodiments of the methods for the preparation of a complex of formula (I) described above, each L₁ is independently an N-containing heteroaryl, —NH₃ or —NHR¹; wherein each R¹ is independently a substituted or unsubstituted moiety selected from alkyl, alkenyl, alkynyl, cycloalkyl, cycloalkyl-alkyl, aryl, aralkyl, heteroaryl, and heteroaralkyl. In some embodiments, each L₁ is the same. In some embodiments, each L₁ is different. In some embodiments, at least one L₁ is —NH₃. In some embodiments, each L₁ is —NH₃.

In some embodiments of the methods for the preparation of a complex of formula (I), the complex is:
(I-A): cis-diammineoxalatochloronitroplatinum(IV);
(I-B): cis-diammineoxalatobromonitroplatinum(IV);
(I-C): cis-diammine(cyclobutane-1,1-dicarboxylato)chloronitroplatinum(IV);
(I-D): cis-diammine(cyclobutane-1,1-dicarboxylato)bromonitroplatinum(IV);

or a pharmaceutically acceptable salt thereof or solvate of the foregoing.

In some embodiments of the methods for the preparation of a complex of formula (II) described above, the donor atoms of the bidentate ligand L₂-L₂ are both N. In some embodiments, one donor atom of the bidentate ligand L₂-L₂ is N and the other donor atom is S. In some embodiments, L₂-L₂ comprises at least one aromatic or non-aromatic cyclic N donor atom (e.g., an N-containing heteroaryl, such as pyridine or imidazole, or an N-containing heterocycle, such as piperidine or piperazine). In some embodiments, L₂-L₂ comprises at least at one aliphatic N donor atom. In some embodiments, L₂-L₂ comprises at least one N donor atom which is an exocyclic amine (e.g., from an aromatic or non-aromatic 5-8 membered ring). In some embodiments, both N donor atoms of L₂-L₂ are exocyclic amines (e.g., from an aromatic or non-aromatic 5-8 membered ring). In some embodiments, L₂-L₂ forms a 5 or 6-membered chelate ring with the platinum atom. In some embodiments, L₂-L₂ forms a 5-membered chelate ring with the platinum atom. In some embodiments, L₂-L₂ forms a 6-membered chelate ring with the platinum atom. In some embodiments, the bidentate ligand L₂-L₂ comprises cycloalkyl or heterocycloalkyl. In some embodiments, the bidentate ligand L₂-L₂ comprises cycloalkyl. In some embodiments, the cycloalkyl is a 5-8 membered cycloalkyl. In some embodiments, the cycloalkyl is cyclohexane. In some embodiments, L₂-L₂ comprises aryl or heteroaryl. In some embodiments, L₂-L₂ comprises a heteroaryl (e.g., imidazole, pyridine, pyrazine, or pyrazine).

In some embodiments of the methods for the preparation of a complex of formula (II), the complex is:
(II-A): (trans-l-1,2-diaminocyclohexane)oxalatochloronitro-platinum(IV);
(II-B): (trans-l-1,2-diaminocyclohexane)oxalatobromonitro-platinum(IV);
(II-C): (1-butyl-2-(aminomethyl)imidazole)oxalatochloronitroplatinum(IV);
(II-D): (2-amino-3-(4-imidazolyl)propionate)oxalato-chloronitroplatinum(IV);
(II-E): (2-amino-3-(4-imidazolyl)methylpropionate) oxalato-chloronitroplatinum(IV);
(II-F): (1-methyl-2-(aminophenylmethyl)imidazole)oxalato-chloronitroplatinum(IV);
(II-G): (1-butyl-2-(methylthiomethyl)imidazole)oxalatochloro-nitroplatinum(IV);
(II-H): (1-methyl-2-(methylthiomethyl)imidazole) oxalatochloro-nitroplatinum(IV);

or a pharmaceutically acceptable salt thereof or solvate of the foregoing.

### Formulations

The platinum(IV) complexes described herein (e.g., any complex of formula I, II, III, or IV) may be used in the preparation of a formulation, such as a pharmaceutical composition or formulation, by combining the platinum(IV) complex(es) described with a pharmaceutical acceptable carrier, excipients, stabilizing agents and/or other agents, which are known in the art, for use in the methods of treatment, methods of administration, and dosage regimes described herein. The formulations may vary or be tailored according to the condition to be treated, the amount of compound to be administered, the condition of the individual, and other variables that will readily be apparent to one of ordinary skill in the art in view of the teachings provided herein. The platinum(IV) complexes may be formulated, for example, as a solid, semi-solid, and liquid dosage forms, such as tablets, pills, powders, liquid solutions or suspensions, suppositories, injectable and infusible solutions, and sprays. The preferred form depends on the intended mode of administration and therapeutic application. The following formulations, additives, and methods are merely exemplary and are in no way limiting.

Additives used with the platinum(IV) complexes described herein (e.g., any complex of formula I, II, III, or IV) include, for example, one or more excipients (e.g., one or more excipients), antioxidants (e.g., one or more antioxidants), stabilizers (e.g., one or more stabilizers), preservatives (e.g., one or more preservatives), pH adjusting and buffering agents (e.g., one or more pH adjusting and/or buffering agents), tonicity adjusting agents (e.g., one or more tonicity adjusting agents), thickening agents (e.g., one or more thickening agents), suspending agents (e.g., one or more suspending agents), binding agents (e.g., one or more binding agents, viscosity-increasing agents (e.g., one or more viscosity-increasing agents), and the like, either alone or together with one or more additional pharmaceutical agents, provided that the additional components are pharmaceutically acceptable for the particular disease to be treated (e.g., cancer). In some embodiments, the formulation may include combinations of two or more of the additional components as described herein (e.g., 2, 3, 4, 5, 6, 7, 8, or more additional components). In some embodiments, the additives include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low melting waxes, ion exchange resins, and the like, as well as combinations of any two or more thereof. Other suitable pharmaceutically acceptable excipients are described in REMINGTON′S PHARMACEUTICAL SCIENCES, Marck Pub. Co., New Jersey 18^{th} edition (1996), and REMINGTON'S THE SCIENCE AND PRACTICE OF PHARMACY, Lippincott Williams & Wilkins, Philadelphia, 20^{th} edition (2003) and 21^{st} edition (2005).

Formulations suitable for oral administration may comprise, for example, (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, (d) suitable emulsions, and (e) powders. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The platinum(IV) complexes can be enclosed in a hard or soft capsule, can be compressed into tablets, or can be incorporated with beverages or food or otherwise incorporated into the diet. Capsules can be formulated by mixing the platinum(IV) complex with an inert pharmaceutical diluent and inserting the mixture into a hard gelatin capsule of the appropriate size. If soft capsules are desired, a slurry of the platinum(IV) complex with an acceptable vegetable oil, light petroleum or other inert oil can be encapsulated by machine into a gelatin capsule.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizing agents, and preservatives. The formulations can be presented in unit-dose or multi-dose sealed containers, such as ampules and vials, and can be stored in a freeze-dried (lyophilized) condition requiring only the addition of the sterile liquid excipient methods of treatment, methods of administration, and dosage regimes described herein (i.e., water) for injection, immediately prior to use. Extemporaneous injection solutions and suspensions can be prepared from sterile powders, granules, and tablets of the kind previously described.

Formulation of the platinum(IV) complex(es) in liquid form (for oral administration, parenteral administration, or otherwise) may have a pH in the range of about 4.5 to about 9.0, including for example pH ranges of any of about 5.0 to about 8.0, about 6.5 to about 7.5, and about 6.5 to about 7.0. In some embodiments, the pH of the composition is formulated to no less than about 6, including for example no less than about any of 6.5, 7, or 8 (e.g., about 8). The formulation can also be made to be isotonic with blood by the addition of a suitable tonicity modifier, such as glycerol.

The platinum(IV) complexes may also be formulated for administration by inhalation. Formulations suitable for aerosol administration which comprise the platinum(IV) complex may include, for example, aqueous and non-aqueous, isotonic sterile solutions, which can contain anti-oxidants, buffers, bacteriostats, and solutes, as well as aqueous and non-aqueous sterile suspensions that can include suspending agents, solubilizers, thickening agents, stabilizing agents, and preservatives, alone or in combination with other suitable components, which can be made into aerosol formulations to be administered via inhalation. These aerosol formulations can be placed into pressurized acceptable propellants, such as dichlorodifluoromethane, propane, nitrogen, and the like. They also can be formulated as pharmaceuticals for non-pressured preparations, such as in a nebulizer or an atomizer.

The platinum(IV) complexes may also be formulated in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The platinum(IV) complexes may also be formulated for topical administration, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

Also provided are unit dosage forms comprising the formulations described herein. These unit dosage forms can be stored in a suitable packaging in single or multiple unit dosages and may also be further sterilized and sealed. For example, the pharmaceutical formulation (e.g., a dosage or unit dosage form of a pharmaceutical formulation) may include (i) a platinum(IV) complex (e.g., a complex of any one of formulas (I)-(IV)) and (ii) a pharmaceutically acceptable carrier. In some embodiments, the pharmaceutical formulation also includes one or more other compounds (or pharmaceutically acceptable salts thereof) that are useful for treating cancer. In various variations, the amount of platinum(IV) complex in the formulation is included in any of the following ranges: about 5 to about 50 mg, about 20 to about 50 mg, about 50 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg. In some embodiments, the amount of platinum(IV) complex in the formulation (e.g., a dosage or unit dosage form) is in the range of about 5 mg to about 500 mg, such as about 30 mg to about 300 mg or about 50 mg to about 200 mg, of the complex. In some embodiments, the carrier is suitable for parental administration (e.g., intravenous administration). In some embodiments, the platinum(IV) complex is the only pharmaceutically active agent for the treatment of cancer that is contained in the formulation.

In some embodiments, are provided dosage forms (e.g., a unit dosage form) for the treatment of cancer comprising (i) a platinum(IV) complex (e.g., a complex of any one of formulas (I)-(IV)), wherein the amount of complex in the unit dosage form is in the range of about 5 mg to about 500 mg, and (ii) a pharmaceutically acceptable carrier. In some embodiments, the amount of platinum(IV) complex in the unit dosage form includes about 30 mg to about 300 mg.

### Kits

Also provided are kits containing materials useful for the treatment of a disease that is responsive to the platinum(IV) complexes (e.g., cancer). The kits may contain a platinum(IV) complex (e.g., any complex of formula I, II, III, or IV) and optionally contain instructions for use (e.g., instructions for preparation and/or administration of a formulation comprising a platinum(IV) complex). Information detailing possible side effects of the formulation, and any other relevant information may also be enclosed. The instructions may be in any suitable format, including, but not limited to, printed matter, videotape, computer readable disk, optical disc or directions to internet-based instructions.

In one aspect, is provided a kit for treating an individual who suffers from or is susceptible to the disease or conditions described herein, comprising a first container comprising a dosage amount of a formulation as disclosed herein, and instructions for use. The container may be any of those known in the art and appropriate for storage and delivery of intravenous formulation. In certain embodiments the kit further comprises a second container comprising a pharmaceutically acceptable carrier, diluent, adjuvant, etc. for preparation of the formulation to be administered to the individual.

In some embodiments, the kits comprise a container with a label. Suitable containers include, for example, bottles, vials, and test tubes. The containers may be formed from a variety of materials such as glass or plastic. The containers may hold a platinum(IV) complex or a formulation of a platinum(IV) complex (e.g., a formulation comprising a platinum(IV) complex and further comprising one or more additional pharmaceutical agents). The label on the container may indicate that the platinum(IV) complex or the formulation is used for treating or suppressing a condition that is responsive to the platinum(IV) complex (e.g., cancer), and may also indicate directions for either in vivo or in vitro use, such as those described herein.

The kit may further include other materials desirable from a commercial and user standpoint, including other buffers, diluents, filters, needles, syringes, and package inserts with instructions for performing any methods described herein. In some embodiments, the kit comprises the container described above and a second container comprising a buffer.

The kits may include additional pharmaceutical agents for use in conjunction with the formulation described herein. In some variations, the additional pharmaceutical agent(s) may be one or more anticancer drug(s). These agents may be provided in a separate form, or mixed with the complexes described herein, provided such mixing does not reduce the effectiveness of either the pharmaceutical agent or formulation described herein and is compatible with the route of administration. Similarly the kits may include additional agents for adjunctive therapy or other agents known to the skilled artisan as effective in the treatment or prevention of the conditions described herein.

Kits may also be provided that contain sufficient dosages of the compounds described herein (including formulations thereof) to provide effective treatment for an individual for an extended period, such as 1-3 days, 1-5 days, a week, 2 weeks, 3, weeks, 4 weeks, 6 weeks, 8 weeks, 3 months, 4 months, 5 months, 6 months, 7 months, 8 months, 9 months or more.

The kits may include the composition as described herein packaged in either a unit dosage form or in a multi-use form. The kits may also include multiple units of the unit dose form. The kits may be used for any of the methods described herein, including, for example, to treat an individual with cancer, or to delay cancer. In certain embodiments the kits may include a dosage amount of at least one formulation as disclosed herein. Kits may also comprise a means for the delivery of the formulation thereof.

### Methods of Treatment

The platinum(IV) complexes of described herein may be used to treat diseases associated with cellular proliferation or hyperproliferation, such as cancers. In some embodiments are provided methods of treating a proliferative disease (e.g., cancer) in an individual, comprising administering to the individual an effective amount of the platinum(IV) complex (e.g., any complex of formula I, II, III, or IV) or a composition comprising an effective amount of the platinum(IV) complex. In some embodiments are provided methods of delaying a proliferative disease (e.g., cancer) in an individual, comprising administering to the individual an effective amount of the platinum(IV) complex or a composition comprising an effective amount of the platinum(IV) complex.

The platinum(IV) complexes described herein may be used to inhibit and/or delay cell proliferation. In some embodiments are provided methods of inhibiting and/or delaying cell proliferation comprising contacting the cells with a platinum(IV) complex (e.g., any complex of formula I, II, III, or IV). In some embodiments are provided methods of inhibiting and/or delaying cell proliferation in an individual, comprising contacting the cells with an effective amount of a platinum(IV) complex (e.g., any complex of formula I, II, III, or IV). In some embodiments, the cell proliferation is undesirable cell proliferations (e.g., cancer cell proliferation).

Examples of cancers that may be treated, inhibited, or delayed by the methods described herein include, but are not limited to, multiple myeloma, renal cell carcinoma, prostate cancer, lung cancer, melanoma, colon cancer, colorectal cancer, ovarian cancer, liver, renal, gastric, and breast cancer.

In some variations, the individual being treated for a proliferative disease has been identified as having one or more of the conditions described herein. Identification of the conditions as described herein by a skilled physician is routine in the art (e.g., via blood tests, X-rays, CT scans, endoscopy, biopsy, etc.) and may also be suspected by the individual or others, for example, due to tumor growth, hemorrhage, ulceration, pain, enlarged lymph nodes, cough, jaundice, swelling, weight loss, cachexia, sweating, anemia, paraneoplastic phenomena, thrombosis, etc. In some embodiments, the individual has been identified as susceptible to one or more of the conditions as described herein. The susceptibility of an individual may be based on any one or more of a number of risk factors and/or diagnostic approaches appreciated by the skilled artisan, including, but not limited to, genetic profiling, family history, medical history (e.g., appearance of related conditions), lifestyle or habits.

In some embodiments, the methods and/or compositions used herein reduce the severity of one or more symptoms associated with proliferative disease (e.g., cancer) by at least about any of 10%, 20%, 30%, 40%, 50%, 60%, 70%, 80%, 90%, 95%, or 100% compared to the corresponding symptom in the same individual prior to treatment or compared to the corresponding symptom in other individuals not receiving the methods and/or compositions.

### Combination Therapy

The platinum(IV) complexes described herein (e.g., any complex of formula I, II, III, or IV) may be formulated and/or administered in conjunction with one or more additional pharmaceutical agents, as described herein and as known in the art, including one or more additional pharmaceutical agents to further reduce the occurrence and/or severity of symptoms and/or clinical manifestations thereof, as well as additional pharmaceutical agents that treat or prevent the underlying conditions, and/or in conjunction with (e.g., prior to, concurrently with, or after) additional treatment modalities. As used herein, the term “additional treatment modality” refers to treatment/prevention of the conditions described herein without the use of a pharmaceutical agent (e.g., surgery, radiotherapy, etc.). Where combinations of pharmaceutical agent(s) and/or additional treatment modality(ies) are used, they may be, independently, administered prior to, concurrently with, or after administration of one or more of the platinum(IV) complexes (or formulation(s) thereof) as described herein.

In some embodiments, the platinum(IV) complexes described herein (e.g., any complex of formula I, II, III, or IV) may be used in combination with one or more additional pharmaceutical agents. The complexes may also be administered in conjunction with (e.g., prior to, concurrently with, or after) agents to alleviate the symptoms associated with either the disease or the treatment regimen. Representative additional pharmaceutical agents include anticancer agents, pre-medication (e.g., corticosteroids, such as dexamethasone, prednisone, prednisolone, etc.), anti-emetics (e.g., antihistamines, such as diphenhydramine), selective 5HT₃ receptor antagonists (e.g. ondansetron), and H₂-receptor antagonists (e.g., cimetidine, ranitidine). Examples of anticancer agents contemplated for combination with the platinum(IV) complexes include, but are not limited to, other platinum-based anti-cancer compounds (e.g., cisplatin, oxaliplatin, carboplatin); vinblastine and/or bleomycin (with or without cisplatin); pemetrexed (as pemetrexed disodium; with or without cisplatin); topotecan (as hydrochloride; with or without cisplatin); paclitaxel (with or without cisplatin); docetaxel (with or without cisplatin); docetaxel (with or without cisplatin); 5-fluorouracil (with or without cisplatin); and capecitabine (with or without another platinum-based regimen, such as cisplatin).

The above additional pharmaceutical agents (e.g., anticancer agents) administered with one or more of the platinum(IV) complexes described herein (e.g., any complex of formula I, II, III, or IV) can be administered at the recommended maximum clinical dosage or at lower doses, such as those indicated in the PHYSICIANS' DESK REFERENCE (PDR) 53rd Edition (1999), or at such therapeutically useful amounts as would be known to one of ordinary skill in the art. Dosage levels of the additional pharmaceutical agents in the formulations may be varied so as to obtain a desired therapeutic response depending on the route of administration, severity of the disease and the characteristics and response of the patient. When administered as a combination, the platinum(IV) complexes can be formulated as separate formulations, which are given at the same time or different times, or the platinum(IV) complex can be given with the additional pharmaceutical agent as a single formulation.

In some embodiments, are provided methods of treating cancer in an individual by administering to the individual an effective amount of a combination of a) a first therapy that comprises a platinum(IV) complex described herein and b) a second therapy useful for treating cancer. In some embodiments, the second therapy includes surgery, radiation, gene therapy, immunotherapy, bone marrow transplantation, stem cell transplantation, hormone therapy, targeted therapy, cryotherapy, ultrasound therapy, and/or photodynamic therapy. It is understood that reference to and description of methods of treating cancer herein is exemplary and that this description applies equally to and includes methods of treating cancer using combination therapy.

The optimal combination of one or more additional pharmaceutical agents and/or one or more additional treatment modalities in conjunction with administration of the platinum(IV) complexes described herein can be determined by an attending physician or veterinarian based on the individual and taking into consideration the various factors effecting the particular individual, including those described herein.

### Dosing and Methods of Administration

The amount of the platinum(IV) complex administered to an individual (such as a human) may vary with the particular formulation, the method of administration, and the particular type of recurrent cancer being treated, and should be sufficient to produce a desirable beneficial effect. The amount administered in order to achieve an effective amount will depend upon a variety of factors, including, for example, the particular condition being treated, the frequency of administration, the particular formulation being administered, the severity of the condition being treated and the age, weight and general health of the individual, the adverse effects experienced by the individual being treated, etc. A pharmaceutical unit dosage chosen may be fabricated and administered to provide a defined final concentration of drug in the blood, tissues, organs, or other targeted region of the body. Determination of an effective amount for a given situation can be readily determined by routine experimentation (e.g., using in vivo animal models) and is within the skill and judgment of the ordinary clinician, particularly in view of the teachings provided herein.

In some embodiments, the amount of the platinum(IV) complex is effective to result in an objective response (such as a partial response or a complete response). In some embodiments, the amount of the platinum(IV) complex is sufficient to result in a complete response in the individual. In some embodiments, the amount of the complex is sufficient to result in a partial response in the individual. In some embodiments, the amount of the complex administered alone is sufficient to produce an overall response rate of more than about any of about 10%, 20%, 30%, 40%, 50%, 60%, 70%, 80%, 90%, or 95% among a population of individuals treated with the complex. Responses of an individual to the treatment of the methods described herein can be determined, for example, based on RECIST or CA-125 level. For example, when CA-125 is used, a complete response can be defined as a return to a normal range value of at least 28 days from the pretreatment value. A partial response can be defined as a sustained over 50% reduction from the pretreatment value.

In some embodiments, the amount of the platinum(IV) complex is sufficient to prolong progress-free survival of the individual (for example as measured by RECIST or CA-125 changes). In some embodiments, the amount of the complex is sufficient to prolong overall survival of the individual. In some embodiments, the amount of the composition is sufficient to produce clinical benefit of more than about any of 10%, 20%, 30%, 40%, 50%, 60%, 70%, 80%, 90%, or 95% among a population of individuals treated with the complex.

In some embodiments, the amount of the platinum(IV) complex is below the level that induces a toxicological effect (i.e., an effect above a clinically acceptable level of toxicity) or is at a level where a potential side effect can be controlled or tolerated when the complex is administered to the individual. In some embodiments, the amount of the complex is close to a maximum tolerated dose (MTD) of the complex following the same dosing regime. In some embodiments, the amount of the complex is more than about any of 80%, 90%, 95%, or 98% of the MTD.

In some embodiments, the amount of the platinum(IV) complex is an amount sufficient to decrease the size of a tumor, decrease the number of cancer cells, or decrease the growth rate of a tumor by at least about any of 10%, 20%, 30%, 40%, 50%, 60%, 70%, 80%, 90%, 95% or 100% compared to the corresponding tumor size, number of cancer cells, or tumor growth rate in the same subject prior to treatment or compared to the corresponding activity in other subjects not receiving the treatment. Standard methods can be used to measure the magnitude of this effect, such as in vitro assays with purified enzyme, cell-based assays, animal models, or human testing.

In some embodiments, the amount of platinum(IV) complex (e.g., platinum(IV) complex in a formulation) is included in any of the following ranges: about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg. In some embodiments, the amount of platinum(IV) complex in the effective amount of the formulation (e.g., a unit dosage faun) is in the range of about 5 mg to about 500 mg, such as about 30 mg to about 300 mg or about 50 mg to about 200 mg. In some embodiments, the concentration of the platinum(IV) complex in the formulation is dilute (about 0.1 mg/ml) or concentrated (about 100 mg/ml), including for example any of about 0.1 to about 50 mg/ml, about 0.1 to about 20 mg/ml, about 1 to about 10 mg/ml, about 2 mg/ml to about 8 mg/ml, about 4 to about 6 mg/ml, about 5 mg/ml. In some embodiments, the concentration of the platinum(IV) complex is at least about any of 0.5 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, or 50 mg/ml.

Exemplary effective amounts of platinum(IV) complex include, but are not limited to, about any of 25 mg/m², 30 mg/m², 50 mg/m², 60 mg/m², 75 mg/m², 80 mg/m², 90 mg/m², 100 mg/m², 120 mg/m², 160 mg/m², 175 mg/m², 180 mg/m², 200 mg/m², 210 mg/m², 220 mg/m², 250 mg/m², 260 mg/m², 300 mg/m², 350 mg/m², 400 mg/m², 500 mg/m², 540 mg/m², 750 mg/m², 1000 mg/m², or 1080 mg/m² of a platinum(IV) complex. In various variations, the composition includes less than about any of 350 mg/m², 300 mg/m², 250 mg/m², 200 mg/m², 150 mg/m², 120 mg/m², 100 mg/m², 90 mg/m², 50 mg/m², or 30 mg/m² of a platinum(IV) complex. In some embodiments, the amount of the platinum(IV) complex per administration is less than about any of 25 mg/m², 22 mg/m², 20 mg/m², 18 mg/m², 15 mg/m², 14 mg/m², 13 mg/m², 12 mg/m², 11 mg/m², 10 mg/m², 9 mg/m², 8 mg/m², 7 mg/m², 6 mg/m², 5 mg/m², 4 mg/m², 3 mg/m², 2 mg/m², or 1 mg/m². In some embodiments, the effective amount of a platinum(IV) complex is included in any of the following ranges: about 1 to about 5 mg/m², about 5 to about 10 mg/m², about 10 to about 25 mg/m², about 25 to about 50 mg/m², about 50 to about 75 mg/m², about 75 to about 100 mg/m², about 100 to about 125 mg/m², about 125 to about 150 mg/m², about 150 to about 175 mg/m², about 175 to about 200 mg/m², about 200 to about 225 mg/m², about 225 to about 250 mg/m², about 250 to about 300 mg/m², about 300 to about 350 mg/m², or about 350 to about 400 mg/m².

In some embodiments of any of the above aspects, the effective amount of a platinum(IV) complex includes at least about any of 1 mg/kg, 2.5 mg/kg, 3.5 mg/kg, 5 mg/kg, 6.5 mg/kg, 7.5 mg/kg, 10 mg/kg, 15 mg/kg, or 20 mg/kg. In various variations, the effective amount of a platinum(IV) complex includes less than about any of 350 mg/kg, 300 mg/kg, 250 mg/kg, 200 mg/kg, 150 mg/kg, 100 mg/kg, 50 mg/kg, 25 mg/kg, 20 mg/kg, 10 mg/kg, 7.5 mg/kg, 6.5 mg/kg, 5 mg/kg, 3.5 mg/kg, 2.5 mg/kg, or 1 mg/kg of a platinum(IV) complex.

Exemplary dosing frequencies include, but are not limited to, weekly without break; weekly, three out of four weeks; once every three weeks; once every two weeks; weekly, two out of three weeks. In some embodiments, the composition is administered about once every 2 weeks, once every 3 weeks, once every 4 weeks, once every 6 weeks, or once every 8 weeks. In some embodiments, the composition is administered at least about any of 1×, 2×, 3×, 4×, 5×, 6×, or 7× (i.e., daily) a week. In some embodiments, the intervals between each administration are less than about any of 6 months, 3 months, 1 month, 20 days, 15, days, 12 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

The administration of the platinum(IV) complex can be extended over an extended period of time, such as from about a month up to about seven years. In some embodiments, the composition is administered over a period of at least about any of 2, 3, 4, 5, 6, 7, 8, 9, 10, 11, 12, 18, 24, 30, 36, 48, 60, 72, or 84 months. In some embodiments, the platinum(IV) complex is administered over a period of at least one month, wherein the interval between each administration is no more than about a week, and wherein the dose of the platinum(IV) complex at each administration is about 0.5 mg/m² to about 250 mg/m², such as about 25 mg/m² to about 150 mg/m² or about 50 mg/m² to about 100 mg/m².

Other exemplary dosing schedules for the administration of the platinum(IV) complex include, but are not limited to, 100 mg/m², weekly, without break; 75 mg/m² weekly, 3 out of four weeks; 100 mg/m², weekly, 3 out of 4 weeks; 125 mg/m², weekly, 3 out of 4 weeks; 125 mg/m², weekly, 2 out of 3 weeks; 130 mg/m², weekly, without break; 175 mg/m², once every 2 weeks; 260 mg/m², once every 2 weeks; 260 mg/m², once every 3 weeks; 180-300 mg/m², every three weeks; 60-175 mg/m², weekly, without break; 20-150 mg/m² twice a week; and 150-250 mg/m² twice a week. The dosing frequency of the complex may be adjusted over the course of the treatment based on the judgment of the administering physician.

The platinum(IV) complexes described herein allow, in some embodiments, infusion of the complex to an individual over an infusion time that is shorter than about 24 hours. For example, in some embodiments, the platinum(IV) complex is administered over an infusion period of less than about any of 24 hours, 12 hours, 8 hours, 5 hours, 3 hours, 2 hours, 1 hour, 30 minutes, 20 minutes, or 10 minutes. In some embodiments, the complex is administered over an infusion period of about 30 minutes.

Any of the platinum(IV) complex described herein can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, sustained continuous release formulation of the composition may be used. In one variation, platinum(IV) complexes can be administered by any acceptable route including, but not limited to, orally, intramuscularly, transdermally, intravenously, through an inhaler or other air borne delivery systems and the like. Additional methods of administration are known in the art.

In some embodiments, the platinum(IV) complexes described herein (e.g., any complex of formula I, II, III, or IV) are administered parenterally (e.g., intravenously). In some embodiments are provided methods of treating cancer comprising parenterally (e.g., intravenously) administering a platinum(IV) complex described herein. Injectable preparations (for example, sterile injectable aqueous or oleaginous suspensions) may be formulated according to the known art using suitable dispersing or wetting agents and suspending agents. The sterile injectable preparation may also be a sterile injectable solution or suspension in a nontoxic parenterally acceptable diluent or solvent, for example, as a solution in propylene glycol. The sterile injectable preparation may also be a sterile powder to be reconstituted using acceptable vehicles prior to administration. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid may be used in the preparation of injectables.

The physiochemical properties (such as stability in vivo) of the platinum(IV) complexes described herein (e.g., a complex of any one of formulas (I)-(IV)) may allow the complexes to be taken orally. In some embodiments, the platinum(IV) complexes or formulations comprising the complexes are suitable for oral administration. The complexes described for use herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, and in other suitable forms.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such formulations may also comprise adjuvants, such as wetting agents, emulsifying and suspending agents, cyclodextrins, and sweetening, flavoring, and perfuming agents.

Also provided are formulations of the platinum(IV) complexes (e.g., a complex of any one of formulas (I)-(IV)) administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

As described herein, the platinum(IV) complexes may be administered with an additional therapeutic agent and/or an additional treatment modalitiy. The dosing frequency of the platinum(IV) complex and the additional therapeutic agent may be adjusted over the course of the treatment based on the judgment of the administering physician. In some embodiments, the platinum(IV) complex and the additional therapeutic agent are administered simultaneously, sequentially, or concurrently. When administered separately, the platinum(IV) complex and the additional therapeutic agent can be administered at different dosing frequency or intervals. For example, the platinum(IV) complex can be administered weekly, while the additional therapeutic agent can be administered more or less frequently. In some embodiments, sustained continuous release formulation of the platinum(IV) complex and/or the additional therapeutic agent may be used. Various formulations and devices for achieving sustained release are known in the art. A combination of the administration configurations described herein can be used.

The present invention will be understood more readily by reference to the following examples, which are provided by way of illustration and are not intended to be limiting of the present invention.

### EXAMPLES

### Example 1

### Synthesis of trans-l-1,2-diaminocyclohexane oxalatochloronitro-platinum(IV)

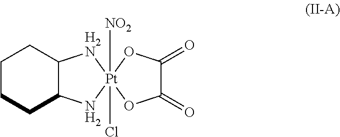

(trans-l-1,2-diaminocyclohexane)oxalatochloronitro-platinum(IV) may be synthesized from (trans-l-1,2-diaminocyclohexane)oxalatoplatinum(II) (oxaliplatin) as a starting material, which may be prepared either by a silver method (such as that described in U.S. Pat. Nos. 4,169,846; 5,290,961; 5,338,874 and 5,420,319); or methods which do not make use of silver described in US 2007/0167643, US 2008/0064895, and WO 2007/085957.

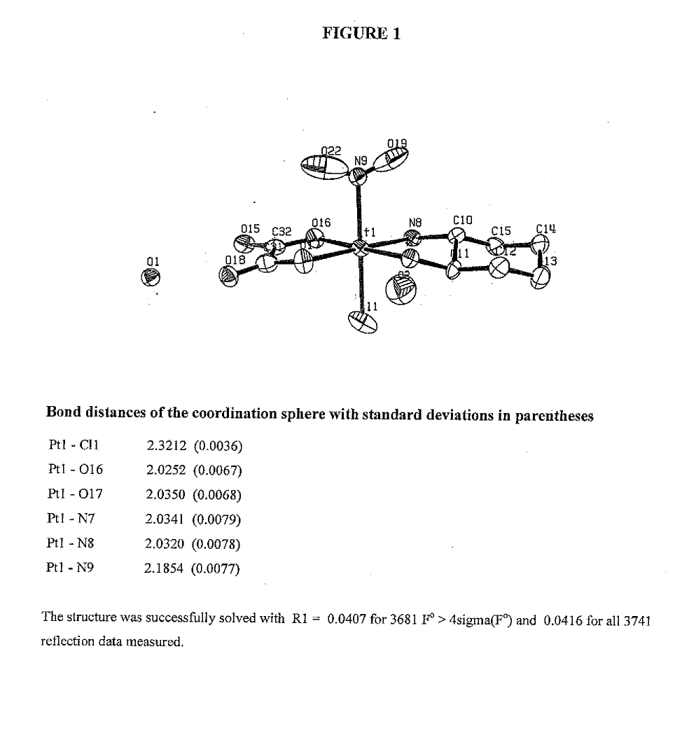

Oxaliplatin (3.24 g) was suspended in a mixture of water (60 mL), containing 1 mole equivalent sodium chloride (0.47 g), and acetone (140 mL) (water:acetone 30:70). Nitrogen dioxide gas was bubbled at a moderate rate through the mixture at room temperature using a source of NO₂. Dissolution of oxaliplatin occurred from the onset of NO₂ addition and was accompanied by a blue-green colouration of the resulting solution. The introduction of NO₂ was discontinued after 75 minutes and the solution left to stir for 10 hr at room temperature where after it assumed a bright yellow colouration. Following evaporation of 75% of the solvent, a yellow solid precipitated. This was washed with three small aliquots of cold water and dried at 60° C. A 75% yield of 95% (verified by hplc) pure product was obtained. Its aqueous solubility is in excess of 20 mg per mL of water from which it can be recrystallized to obtain a higher degree of purity. The solid complex is stable up to 210° C. ESI-MS: 496.8 [M+NH₄]⁺, 479.9 [M+H]⁺, 477.9 [M−]⁻. Product purity verified by hplc on a Phenomenx Curosil PFP column (250×4.6 mm 5 μm); 1.0 ml/min; 2.5% acetonitrile, UV 210. A crystallographic structure of the dihydrate of (trans-l-1,2-diaminocyclohexane)oxalatochloronitro-platinum(IV) is shown in FIG. 1.

### Example 2

### Synthesis of (trans-l-1,2-diaminocyclohexane)oxalatobromonitro-platinum(IV)

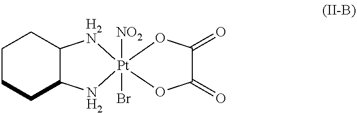

0.815 g (2.05 mmol) of oxaliplatin (trans-(1R,2R)-(−)-1,2-Diamminocyclohexane-oxalatoplatinum(II)) was suspended in 15 mL distilled water and 36 mL acetone (30:70; water:acetone mixture). 1 molar equivalent (0.211 g; 2.05 mmol) sodium bromide was dissolved in a portion of the aforementioned 15 mL distilled water and added to the aforementioned mixture. A milky white suspension was formed and immediately reacted at room temperature with NO₂ gas. The reaction continued for 37 min, upon which a light green solution was obtained and the supply of NO₂ gas terminated, the reaction vessel was covered with foil and allowed to stir overnight at room temperature. After 18 h, a lime green suspension was present. Air was bubbled through this reaction mixture until the original volume was reduced to 25%. The resulting greenish-yellow precipitate was filtered, washed with 3 small aliquots of distilled water and finally dried overnight at 50° C. Yield: 74%, Purity: 93%. Further purification of the final product can be obtained through recrystallization from hot acetone. ESI-MS: 521.7 [M+H]⁺. Product purity verified by hplc on a Phenomenx Curosil PFP column (250×4.6 mm 5 μm); 1.0 ml/min; 2.5% acetonitrile, UV 210.

### Example 3

### Synthesis of cis-diammineoxalatochloronitroplatinum(IV)

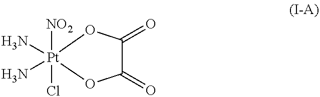

cis-Diammineoxalatoplatinum(II) (0.54 g; preparable with silver and oxalate ions using analogous methods described in U.S. Pat. Nos. 4,169,846; 5,290,961; 5,338,874 and 5,420,319) was suspended in a mixture of water (18 mL) containing one mole equivalent of NaCl (0.11 g), and acetone (42 mL) (water:acetone 30:70). Nitrogen dioxide gas was bubbled at a moderate rate through the mixture at room temperature using a source of NO₂. Dissolution of cis-diammineoxalatoplatinum(II) occurred from the onset of NO₂ addition and was accompanied by a blue-green colouration of the resulting solution. The introduction of NO₂ was discontinued after 80 minutes and the solution left to stir at room temperature overnight where after a yellow suspension resulted. The residual solvent was evaporated under vacuum and the resulting solids were subsequently washed with ethyl acetate (3×15 mL). A final wash with water (3×10 mL) produced 0.339 g of product, which was 94% pure following recrystallization. ESI-MS: 416.8 [M+NH₄]⁺, 442.6 [M+HCOO]⁻, 397.9 [M+H]⁺, 396.9 [M−H]⁻. Product purity was verified by hplc using a YMC C18-hydrosphere column (250×4.6 mm 5 μm); 1.0 ml/min; 20% acetonitrile; UV 210. Water solubility: 2.4 mg/mL.

### Example 4

### Synthesis of (1-butyl-2-(aminomethyl)imidazole) oxalatochloronitroplatinum(IV)

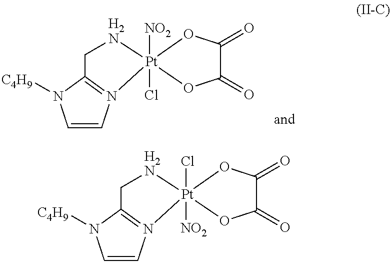

(1-Butyl-2-(aminomethyl)imidazole)oxalatoplatinum(II) (0.25 g; preparable using silver and oxalate ions as described above) was suspended in a mixture of water (10 mL), containing one mole equivalent of NaCl (0.034 g), and acetone (50 mL) (water: acetone 17:83). NO₂ gas, carried by a stream of nitrogen, was bubbled through the suspension. Dissolution of the suspended solids occurred in 1 hr to yield a yellow solution. The NO₂ stream was stopped and the solution stirred in an open vessel overnight at room temperature. The following morning, the light yellow solids which had precipitated during the evaporation of solvent overnight were filtered off, washed with water, and dried at 60° C. (0.21 g). The solid was subsequently dissolved in pure acetone, filtered, and concentrated under vacuum. Crystallization occurred during evaporation. The crystalline product was filtered off, washed with water and dried at 60° C. (0.12 g, 40% yield). ESI-MS: 535.7 [M+NH₄]⁺, 516.9 [M+]⁺.

### Example 5

### Synthesis of (2-amino-3-(4-imidazolyl)methylpropionate)oxalato-chloronitroplatinum(IV)

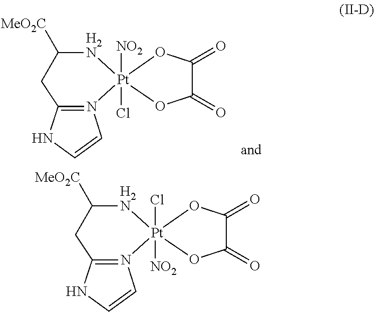

(2-Amino-3-(4-imidazolyl)methylpropionate)oxalatoplatinum(II) (0.44 g; preparable with silver and oxalate ions using analogous methods described in U.S. Pat. Nos. 4,169,846; 5,290,961; 5,338,874 and 5,420,319 with the methyl ester of histidine (2-amino-3-(4-imidazolyl)methylpropionate) as the diamine chelate) was suspended in a mixture of water (17 mL), containing one mole equivalent of NaCl (0.056 g), and acetone (41 mL) (water:acetone 30:70). NO₂ gas, carried by a stream of nitrogen, was bubbled through the suspension. The reaction mixture proceeded through a green intermediary phase to a fine yellow suspension. The flow of NO₂ was terminated after 110 minutes and the mixture stirred overnight in an open vessel at room temperature. The precipitated yellow solids, which formed during the evaporation of the acetone overnight, were subsequently washed with small aliquots of water and dried to yield 0.20 g (37% yield) of the target compound having a purity of 90%. Product purity verified by hplc using a YMC Hydrosphere (C18) (250×4.6 5 μm); 1.0 ml/min, 20% acetonitrile, UV210.

### Example 6

### Synthesis of (1-butyl-2-(methylthiomethyl)imidazole)oxalatochloro-nitroplatinum(IV)

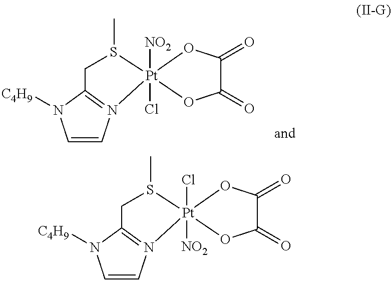

The precursor complex, (1-butyl-2-(methylthiomethyl)imidazole) oxalatoplatinum(II), was synthesized using silver and oxalate ions in a procedure analogous to that described in U.S. Pat. Nos. 4,169,846; 5,290,961,338,874 and 5,420,319. The synthesis of the N—S chelating ligand, 1-methyl-2-(methylthiomethyl)imidazole, is described in WO2006/024897.

(1-Butyl-2-(methylthiomethyl)imidazole)oxalatoplatinum(II) (0.30 g) was suspended in a mixture of water (12 mL), containing one mole equivalent of NaCl (0.037 g), and acetone (28 mL) (water:acetone 30:70). Nitrogen dioxide gas, carried by a stream of nitrogen, was bubbled at a moderate rate through the mixture at room temperature. The flow of NO₂ was discontinued once a homogenous green solution was obtained and was left to stir at room temperature overnight where after it assumed a bright yellow colouration. The solvent was evaporated under vacuum and the resulting solid residue washed with ether and dried. ESI-MS: 547.7 [M−H]⁻

### Example 7

### Anticancer Activity of (trans-l-1,2-diaminocyclohexane)oxalatochloronitro-platinum(IV) (Complex II-A)

The anticancer activity of (trans-l-1,2-diaminocyclohexane)oxalate chloronitro-platinum(IV) (II-A) has been compared with known antitumor agents. In Table 1, the IC50 values as obtained for Hela, HT29 and MCF7 cancer cells are depicted. All Dose response curves were prepared for each of the chosen complexes, as well as positive controls, in order to obtain IC50 values. The concentrations used were 100, 50, 25, 10, 5 and 1 μM. IC50 values were calculated from the log-dose response curves using GraphPad Prism 4.

TABLE 1
Comparison between the IC50 values (μM) of
(trans-l-1,2-diaminocyclohexane)oxalate chloronitro-
platinum(IV) (II-A) and two positive controls:
oxaliplatin and cisplatin.

| Complex | HeLa | HT29 | MCF7 |
|---|---|---|---|
| Cisplatin | 10.67 | 8.11 | 14.10 |
| Oxaliplatin | 11.92 | 12.3 | 6.21 |
| (II-A) | 4.81 | 3.07 | 5.86 |

### Example 8

### Anticancer Activity of (trans-l-1,2-diaminocyclohexane)oxalatobromonitro-platinum(IV) (Complex I-B)

The anticancer activity of (trans-l-1,2-diaminocyclohexane) oxalatobromonitro-platinum(IV) (complex II-B) has been compared to cisplatin against two cell lines for 48 h at 50 uM. Results are shown in Table 2.

TABLE 2
Percent inhibition of HeLa and MCF7 cells.

| Complex | HeLa | MCF7 |
|---|---|---|
| Cisplatin | 86 | 72 |
| (I-B) | 83 | 73 |

### Example 9

### Anticancer activity of cis-diammineoxalatochloronitroplatinum(IV) (Complex I-A)

The anticancer activity of cis-diammineoxalatochloronitroplatinum(IV) (complex I-A) has been compared to cisplatin against two cell lines for 48 h at 77 uM. Results are shown in Table 3.

TABLE 3
Percent inhibition of HeLa and MCF7 cells.

| Complex | HeLa | MCF7 |
|---|---|---|
| Cisplatin | 87 | 76 |
| (I-A) | 84 | 74 |

### Example 10

### Stability data of (trans-l-1,2-diaminocyclohexane)oxalatochloronitro-platinum(IV) (Complex II-A)

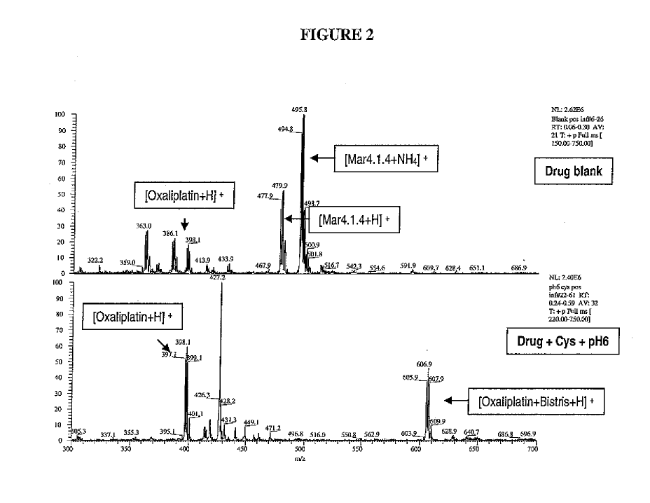

Upon addition of cysteine to complex II-A: (trans-l-1,2-diaminocyclohexane) oxalatochloronitro-platinum(IV)), a reduction pattern was observed, wherein oxaliplatin is formed (see FIG. 2). Positive ions may be attributed to compound II-A (m/z 478 and 495) and are completely replaced by those of oxaliplatin (m/z 397) following treatment. This observation implies that certain platinum(IV) complexes described herein are capable of converting to their corresponding platinum(II) complexes.

1. A platinum complex of formula (I) or (II): 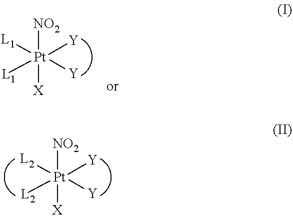
wherein each L₁ is independently a monodentate nitrogen donor ligand;
L₂-L₂ is a bidentate ligand which forms a 5-8 membered chelate ring with the platinum atom and wherein the donor atoms of the bidentate ligand are each independently N or S;
Y—Y is a dicarboxylate linked to the platinum atom through the terminal oxygen atoms;
and X is a halide, —ONO₂, or a carboxylate linked through the oxygen atom;
or a pharmaceutically acceptable salt thereof or solvate of the foregoing.

2. The platinum complex of claim 1, wherein the dicarboxylate is a C₂-C₇ dicarboxylate.

3. The platinum complex of claim 2, wherein the dicarboxylate is an oxalate.

4. The platinum complex of claim 1, of the formula: 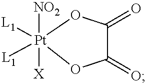
wherein X is a halide.

5. The platinum complex of claim 4, wherein each L₁ is the same.

6. The platinum complex of claim 4, wherein each L₁ is different.

7. The platinum complex of claim 4, wherein at least one L₁ is NH₃.

8. The platinum complex of claim 1, of the formula: 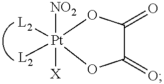
wherein X is a halide.

9. The platinum complex of claim 8, wherein the donor atoms of the bidentate ligand L₂-L₂ are both N.

10. The platinum complex of claim 8, wherein one donor atom of the bidentate ligand L₂-L₂ is N and the other donor atom is S.

11. The platinum complex of claim 8, wherein at least one N donor atom is aromatic.

12. The platinum complex of claim 11, wherein the N donor atom is from a substituted or unsubstituted imidazole.

13. The platinum complex of claim 8, wherein the bidentate ligand L₂-L₂ forms a 5 or 6-membered chelate ring with the platinum atom.

14. The platinum complex of claim 8, wherein the bidentate ligand L₂-L₂ comprises cycloalkyl or heterocycloalkyl.

15. The platinum complex of claim 8, wherein the bidentate ligand L₂-L₂ comprises aryl or heteroaryl.

16. The platinum complex of claim 1, wherein the complex is:
cis-diammineoxalatochloronitroplatinum(IV);
cis-diammineoxalatobromonitroplatinum(IV);
cis-diammine(cyclobutane-1,1-dicarboxylato)chloronitroplatinum(IV);
cis-diammine(cyclobutane-1,1-dicarboxylato)bromonitroplatinum(IV);
(trans-l-1,2-diaminocyclohexane)oxalatochloronitro-platinum(IV);
(trans-l-1,2-diaminocyclohexane)oxalatobromonitro-platinum(IV);
(1-butyl-2-(aminomethyl)imidazole)oxalatochloronitroplatinum(IV);
(2-amino-3-(4-imidazolyl)propionate)oxalato-chloronitroplatinum(IV);
(2-amino-3-(4-imidazolyl)methylpropionate) oxalato-chloronitroplatinum(IV);
(1-methyl-2-(aminophenylmethyl)imidazole)oxalato-chloronitroplatinum(IV);
(1-butyl-2-(methylthiomethyl)imidazole)oxalatochloro-nitroplatinum(IV);
(1-methyl-2-(methylthiomethyl)imidazole) oxalatochloro-nitroplatinum(IV);
or a pharmaceutically acceptable salt thereof or solvate of the foregoing.

17. The platinum complex of claim 1, having the structure: 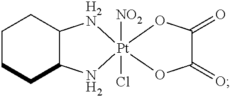
or a pharmaceutically acceptable salt thereof or solvate of the foregoing.

18. The platinum complex of claim 1, having the structure: 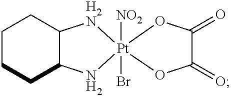
or a pharmaceutically acceptable salt thereof or solvate of the foregoing.

19. The platinum complex of claim 1, having the structure: 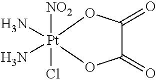
or a pharmaceutically acceptable salt thereof or solvate of the foregoing.

20. The platinum complex of claim 1, wherein the complex has increased stability relative to the corresponding platinum(II) complex lacking X and NO₂.

21. A formulation comprising a platinum complex of claim 1 and a pharmaceutically acceptable carrier.

22. The formulation comprising a platinum complex of claim 16 and a pharmaceutically acceptable carrier.

23. The formulation of claim 22, wherein the platinum complex is cis-diammineoxalatochloronitroplatinum(IV); or a pharmaceutically acceptable salt thereof or solvate of the foregoing.

24. The formulation of claim 22, wherein the platinum complex is cis-diammine(cyclobutane-1,1-dicarboxylato)chloronitroplatinum(IV); or a pharmaceutically acceptable salt thereof or solvate of the foregoing.

25. The formulation of claim 22, wherein the platinum complex is cis-diammine(cyclobutane-1,1-dicarboxylato)bromonitroplatinum(IV); or a pharmaceutically acceptable salt thereof or solvate of the foregoing.

26. A substantially pure form of a platinum complex of claim 1.

27. A method of treating a proliferative disease in an individual, comprising administering to the individual an effective amount of a platinum complex of claim 1.

28. The method of claim 27, wherein the proliferative disease is cancer.

29. The method of claim 28, wherein the cancer is a solid tumor.

30. The method of claim 28, wherein the cancer is selected from the group consisting of colorectal cancer, multiple myeloma, renal cell carcinoma, prostate cancer, lung cancer, melanoma, ovarian cancer, and breast cancer.

31. The method of claim 30, wherein the cancer is colorectal cancer.

32. The method of claim 27, wherein the complex is administered parenterally.

33. The method of claim 27, wherein the complex is administered orally.

34. The method of claim 27, wherein the platinum complex is:
cis-diammineoxalatochloronitroplatinum(IV);
cis-diammineoxalatobromonitroplatinum(IV);
cis-diammine(cyclobutane-1,1-dicarboxylato)chloronitroplatinum(IV);
cis-diammine(cyclobutane-1,1-dicarboxylato)bromonitroplatinum(IV);
(trans-l-1,2-diaminocyclohexane)oxalatochloronitro-platinum(IV);
(trans-l-1,2-diaminocyclohexane)oxalatobromonitro-platinum(IV);
(1-butyl-2-(aminomethyl)imidazole)oxalatochloronitroplatinum(IV);
(2-amino-3-(4-imidazolyl)propionate)oxalato-chloronitroplatinum(IV);
(2-amino-3-(4-imidazolyl)methylpropionate) oxalato-chloronitroplatinum(IV);
(1-methyl-2-(aminophenylmethyl)imidazole)oxalato-chloronitroplatinum(IV);
(1-butyl-2-(methylthiomethyl)imidazole)oxalatochloro-nitroplatinum(IV);
(1-methyl-2-(methylthiomethyl)imidazole) oxalatochloro-nitroplatinum(IV);
or a pharmaceutically acceptable salt thereof or solvate of the foregoing.

35. The method of claim 34, wherein the platinum complex is:
cis-diammineoxalatochloronitroplatinum(IV); or a pharmaceutically acceptable salt thereof or solvate of the foregoing.

36. The method of claim 34, wherein the platinum complex is:
cis-diammine(cyclobutane-1,1-dicarboxylato)chloronitroplatinum(IV); or a pharmaceutically acceptable salt thereof or solvate of the foregoing.

37. The method of claim 34, wherein the platinum complex is:
cis-diammine(cyclobutane-1,1-dicarboxylato)bromonitroplatinum(IV); or a pharmaceutically acceptable salt thereof or solvate of the foregoing.

38. A method of inhibiting proliferation of cells, comprising contacting the cells with a platinum complex of claim 1.

39-40. (canceled)

41. A method for the preparation of a platinum complex of claim 1, comprising reacting a platinum(II) complex in a suitable solvent, with a halide in anionic form or carboxylate in anionic form, and NO₂.

42. A method for the preparation of a platinum complex of formula (I): 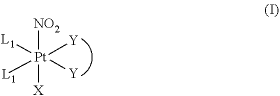
wherein each L₁ is independently a monodentate nitrogen donor ligand;
Y—Y is a dicarboxylate linked to the platinum atom through the terminal oxygen atoms; and X is a halide, —ONO₂, or a carboxylate linked through the oxygen atom; or a pharmaceutically acceptable salt thereof or solvate of the foregoing;
comprising reacting in a suitable solvent a platinum(II) complex of formula (I-P): 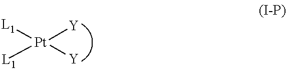
wherein each L₁ and Y—Y is as defined above;
with NO₂ and a halide anion, a nitrate anion, or a carboxylate anion; to form a complex of formula (I), or a pharmaceutically acceptable salt thereof or solvate of the foregoing.

43. A method for the preparation of a platinum complex of formula (II): 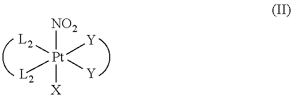
wherein L₂-L₂ is a bidentate ligand which forms a 5-8 membered chelate ring with the platinum atom and wherein the donor atoms of the bidentate ligand are each independently N or S; Y—Y is a dicarboxylate linked to the platinum atom through the terminal oxygen atoms; and X is a halide, —ONO₂, or a carboxylate linked through the oxygen atom; or a pharmaceutically acceptable salt thereof or solvate of the foregoing;
comprising reacting in a suitable solvent a platinum(II) complex of formula (II-P): 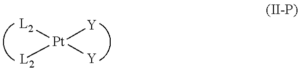
wherein L₂-L₂ and Y—Y is as defined above;
with NO₂, and a halide anion, a nitrate anion, or carboxylate anion; to form a complex of formula (II), or a pharmaceutically acceptable salt thereof or solvate of the foregoing.
